(19) United States
(12) Patent Application Publication
(10) Pub. No.: US 2005/0014676 A1
(43) Pub. Date: Jan. 20, 2005

(54) Methods for delaying or inducing labor

(75) Inventors: Yunping Li, West Roxbury, MA (US); Kathleen Morgan, Newton Centre, MA (US)

Correspondence Address: Kevin M. Farrell;Pierce Atwood, Suite 350, One New Hampshire Avenue, Portsmouth, NH, 03801, US

(73) Assignee: Boston Biomedical Research Institute, Watertown, MA (US)

(21) Appl. No.: 10/620,317

(22) Filed: Jul. 15, 2003

(51) Int. Cl. A61K 38/17; A61K 31/567

(52) U.S. Cl. 514/2; 514/179

(57) ABSTRACT

Disclosed herein are novel methods for preventing or delaying preterm uterine contractions in a pregnant mammal. Also disclosed are novel methods for inducing uterine contractions in a pregnant mammal. In each case, control of uterine smooth muscle contractility is achieved by specifically targeting the MAPK ERK signal transduction cascade. Inactivation of the cascade is disclosed as a novel method for delaying labor. Activation of the MAPK ERK pathway is disclosed as a novel method for inducing labor.

### GOVERNMENT SUPPORT

This invention was made with Government support under NHLBI/NIH grants HL31704 and HL42293 to K. G. Morgan and NIGMS/NIH GM07592 and the Foundation for Anesthesia Education and Research grant to Y. Li. The government has certain rights in the invention.

### BACKGROUND OF THE INVENTION

In the U.S., approximately 10% of all births are premature. Premature birth accounts for approximately 75% of all neonatal death and morbidity including long-term disabilities. However, at present, there is no truly safe and effective agent approved in the U.S. for the selective suppression of preterm uterine contractions. A greater understanding of the mechanisms of uterine contraction during preterm labor and term labor is solely needed for more effective and specific therapeutics to be developed.

Caldesmon (CaD) is an actin binding protein that inhibits actin activated ATPase activity in vitro and has been proposed to be a major regulator of smooth muscle actin-myosin interactions. In vitro, either the binding of calmodulin or phosphorylation of CaD can reverse the inhibition. It is clear that the extracellular signal-regulated kinase (ERK) can phosphorylate CaD in vitro. However, whether ERK regulates the inhibitory action of CaD and muscle contractility in vivo remains a controversial area. It has been difficult to determine a clear-cut, cause and effect relationship between ERK activity and regulation of contractility in a physiological setting.

The ERK pathway is known to be activated during oxytocin-induced contraction and during prostaglandin F2alpha-induced contraction of pregnant rat myometrium. Furthermore, spontaneous term labor is associated with a basal activation of ERK2 and subsequent phosphorylation of CaD that persists in vitro. Thus, the change in ERK/CaD signaling may contribute to the change in uterine contractility during the initiation of parturition. The currently available ERK inhibitors are only effective when used as pretreatment to prevent the initial activation of ERK. Thus, it has not been possible by simple in vitro experiments to determine whether there is a cause and effect relationship between gestation-dependent activation of the ERK/CaD pathway and regulation of uterine contractility. The in vivo effect of ERK inhibition on modulating smooth muscle contractility has not been previously explored.

### SUMMARY OF THE INVENTION

The present invention relates to methods for controlling uterine smooth muscle contractility via specifically targeting the MAPK ERK signal transduction cascade. Inactivation of the cascade is disclosed as a novel method for delaying labor. Activation of the cascade is disclosed as a novel method for inducing labor.

The present invention relates to a method for preventing and/or delaying labor by administering to a pregnant mammal a compound which prevents phosphorylation, and hence activation, of ERK. The MEK inhibitor U0126 prevents phosphorylation of ERK and delays RU486-induced labor in a statistically significant manner. Remarkably, administration of U0126 resulted in no apparent signs of toxicity to either mother or offspring. The present invention also relates to a method for inducing labor by administering to a pregnant mammal a compound which activates kinase activity, thereby resulting in increased levels of phosphorylated, and hence activated, ERK in the pregnant mammal.

Also disclosed are methods for delaying or inducing labor by altering the levels of the phosphorylated downstream targets of ERK, caldesmon (CaD) and/or uterine myosin light chain 20 (LC20), during pregnancy. In a preferred embodiment, the levels of phosphorylated CaD and/or uterine LC20 are altered by administering an inhibitor of ERK kinase. A decrease in the levels of phosphorylated CaD and/or uterine LC20 would result in the prevention of labor, whereas an increase in the levels of phosphorylated CaD and/or uterine LC20 would result in the induction of labor.

Administration of a compound which activates or inhibits the activity of phosphatases on ERK signal transduction components is disclosed as a method for delaying or inducing labor, respectively. Administration of a compound which inhibits or activates the binding of caldesmon to calmodulin is also disclosed as a method for delaying or inducing labor, respectively.

### BRIEF DESCRIPTION OF THE DRAWINGS

FIG. 1 represents the temporal sequence for rat injections relative to gestational stage. Rats in the RU group received an RU486 injection at 10 am of day 19 of pregnancy for the induction of preterm labor. U0+RU rats also received the same injection of RU486, but in addition were treated with U0126, every 8 hours, starting at 7 am of day 18 of pregnancy until the onset of labor. Sham group rats underwent a similar protocol as U0+RU group, but were injected with vehicle (DMSO and sunflower seed oil) instead.

FIG. 2 illustrates that RU486 induces a tightly controlled model of preterm labor in rats. A. Histogram of labor onset times during spontaneous labor. Average gestational length=22.93±0.42 day (Mean±SD, n-=13). B. RU486-induced preterm labor histogram. Average gestational length=20.35±0.02 day (Mean±SD, n=6).

FIG. 3 demonstrates the increased in vitro myometrial contractility in RU486 treated rats. Area under curve (AUC) is the integral of the force trace over 15 min and is normalized to tissue dry weight. Spontaneous labor (SL), RU486-induced preterm labor (RU), U0126 treated group (U0+RU). **p<0.001 compared to sham group, n=6−8 in each group.

FIG. 4 demonstrates that ERK2 is phosphorylated at the onset of RU486-induced preterm labor and spontaneous labor in rats. A. Myometrial ERK2 protein levels are unchanged (p=0.23) during pregnancy and labor in all groups compared to nonpregnant (NP) uterine smooth muscle. B. ERK2 phosphorylation was normalized by ERK2 protein level as measured by western blot and expressed as a ratio, p-ERK2/ERK2. NP: nonpregnant; SL: spontaneous labor; RU: RU486-induced preterm labor; U0+RU: U0126 treated group. ** p<0.01 vs. NP; ♦♦ p<0.01 vs. sham group; ♦p<0.05 vs. sham group. n=6−8 in each group.

FIG. 5 demonstrates that high molecular weight caldesmon (h-CaD) protein content (Panel A) is increased during pregnancy and that h-CaD (Panel B) is phosphorylated with the onset of spontaneous labor and RU486-induced preterm labor. Phosphorylated h-CaD is normalized to h-CaD protein level. *p<0.05 and **p<0.01 compared to nonpregnant (NP) group; ♦p<0.05 and ♦♦p<0.01 compared to sham group. n=6−8 in each group.

FIG. 6 demonstrates that U0126 delays RU486 induced preterm labor in rats. The experimental protocol is as in FIG. 1. RU486 was administered at 10 am of day 19 of pregnancy in both RU and U0+RU groups. Administration of U0126 was started on day 18 of pregnancy, continuing until the labor commenced. The average delivery time is significantly different between RU486 group (22.25±0.24 hr) and U0126 treated group (25.18±0.64 hr), p=0.0016, n=6 in each group.

### DETAILED DESCRIPTION OF THE INVENTION

The present invention relates, in one aspect, to methods for preventing and/or delaying preterm uterine contractions in a pregnant mammal. More specifically, the present invention relates to methods for preventing and/or delaying labor by administration of a compound which prevents activation of a mitogen activated protein kinase (MAPK). In a preferred embodiment, the MAPK whose activation is prevented is the extracellular signal-regulated kinase (ERK).

MAPKs are members of a signal transduction cascade. MAPKs phosphorylate specific serines and threonines of target substrates. This phosphorylation acts as a molecular switch for turning on the activity of target substrates. The activities of MAPKs are controlled by a similar mechanism in which MAPKs are themselves phosphorylated by MAPK kinases (MAPKKs). It can readily be deduced that the activity of a MAPK target substrate can thus be controlled at various levels throughout the signal transduction cascade. Identical downstream physiological consequences can be achieved independently via alteration of any single part of a MAPK signal transduction cascade.

MAPKs regulate a variety of cellular processes ranging from gene expression, mitosis, movement, and metabolism to programmed death. A number of MAPKs have been identified, each with varying substrate specificities. By targeting the activity of a specific MAPK, or a component of a specific MAPK pathway, selectivity of a desired therapeutic result can be achieved while minimizing potential adverse side effects. Because MAPK/ERK kinases (MEKs) typically phosphorylate only one or two MAPKs, they are particularly attractive targets for therapeutic intervention.

The present invention provides the first in vivo evidence for the involvement of the ERK MAPK in smooth muscle regulation. The present invention relates to methods for controlling uterine smooth muscle contractility by specifically targeting the MAPK ERK signal transduction cascade. Administration of a compound which prevents phosphorylation, and hence activation, of ERK prevents preterm uterine contractions.

One of the main findings of the present invention is that U0126, an inhibitor of the MAPKK members MEK-1 and MEK-2, delays RU486-induced preterm labor in a statistically significant manner. Remarkably, administration of U0126 at doses sufficient to delay preterm labor resulted in no apparent signs of toxicity to either mother or offspring. In comparison to tocolytics known in the art, the magnitude of the delay of preterm labor by U0126 is similar to that caused by L-366509, a non-peptide oxytocin receptor antagonist and that caused by diclofenac, a cyclooxygenase inhibitor in rats. The present invention, however, would likely have a distinct temporal advantage over the use of oxytocin receptor antagonists, which are known in the art. Administration of a compound targeting the ERK pathway or other components of said pathway would likely be effective at earlier gestational stages than oxytocin receptor antagonists. These findings are of considerable interest at the clinical level as U0126, or other MEK inhibitors could be administered safely and effectively in delaying preterm labor.

Caldesmon (CaD) is one of the downstream targets of ERK, which is known in the art. ERK is known to phosphorylate CaD in vitro at a C-terminal serine residue. At the onset of labor, an increase in the levels of phosphorylated ERK has been correlated with a resultant increase in the levels of phosphorylated CaD in vivo. One of the inherent consequences of administering a compound which decreases the levels of phosphorylated ERK in a pregnant mammal would likely be a resulting decrease in the levels of phosphorylated caldesmon (CaD) in the pregnant mammal, both cases relative to an otherwise matched pregnant mammal to which the compound has not been administered. An “otherwise matched pregnant mammal” is one who is of the same species, size, weight, age, and gestational stage as the pregnant mammal to which the compound has been administered, the gestational stage defined relative to labor. It is likely that the physiological consequence of preventing or delaying preterm uterine contractions by administration of said compound arises indirectly from affecting the levels of phosphorylated ERK and directly from affecting the levels of phosphorylated CaD. Administration of a compound which targets phosphorylated CaD directly would likely have the same effect on labor as administration of a compound which targets the aforementioned upstream signaling events.

ERK is not the only kinase known in the art to phosphorylate CaD. It is likely that the phosphorylated state of CaD is the determinant of its activity in smooth muscle contraction. Two other kinases, p21-activated kinase (PAK) and calmodulin (CaM) kinase II, are known in the art to phosphorylate CaD. Both PAK and CaM kinase 11 phosphorylate CaD at ERK-independent phosphorylation sites. It is likely that administration of a compound which inhibits the kinase activity of PAK and/or CaM kinase II would likely decrease the phosphorylated levels of caldesmon, thereby preventing or delaying labor in a pregnant mammal.

The downstream effect of inhibiting MEK-1 and MEK-2, and hence decreasing the levels of phosphorylated ERK may be mimicked by a phosphorylation-independent mechanism known to those in the art. ERK-dependent phosphorylation of CaD has been reported to diminish the actin affinity of CaD, alleviate CaD inhibition on actomyosin ATPase activity, and increase motility in in vitro motility assays. Cross linking studies have shown that binding of Ca²+/CaM to CaD likewise decreases the binding of CaD to actin in vitro, suggesting that the activity of Ca²+/CaM-bound CaD would mimic that of the phosphorylated CaD. Administration of a compound which would target this mechanism and inhibit the binding of CaM to CaD would likely be effective in preventing or delaying preterm uterine contractions in a pregnant mammal.

Known in the art are protein phosphatases which remove phosphates that were initially transferred to protein substrates by kinases. Protein phosphatases reverse the phosphorylation state, and hence activation state, of said substrates. Administration of a compound which activates a phosphatase could result in decreased levels of phosphorylated MAPKK, phosphorylated ERK and/or phosphorylated CaD, thereby inhibiting labor in a pregnant mammal. Examples of phosphatases known in the art are an ERK phosphatase and a CaD. phosphatase.

The present invention also relates to a method for preventing or delaying preterm uterine contractions in a pregnant mammal by targeting the levels of uterine phosphorylated myosin light chain (LC20) protein. An increase in the levels of uterine phosphorylated LC20 is associated with the activation of ERK and the onset of labor. Administration of a compound which inhibits kinase activity, thereby resulting in a decrease in the levels of uterine phosphorylated LC20, may be used in the prevention of preterm labor. In a preferred embodiment, said kinase activity which is inhibited is the kinase activity of MAPKK, and the preferred inhibitor is U0126. Inhibitors of other kinases may be administered with regard to the present invention for decreasing levels of uterine phosphorylated LC20. Inhibitors of ERK would also decrease the levels of uterine phosphorylated LC20. Administration of a RhoA/Rho-kinase inhibitor prevents LC20 phosphorylation and oxytocin-induced labor, as is known in the art.

Administration of a compound which would decrease uterine levels of ERK would also likely prevent preterm uterine contractions. A decrease in uterine ERK protein expression, for example, would cause a decrease in the amount of ERK available for phosphorylation by a kinase. The result would be an in an inherent decrease in the levels of phosphorylated, and hence activated, ERK.

The present invention also relates to methods for inducing uterine contractions in a pregnant mammal. The methods disclosed herein, described for inducing uterine contractions, may be utilized for the treatment of full-term dysfunctional labor or conversely for the induction of early labor. Induction of labor at any time during pregnancy might be achieved by affecting the MAPK signal transduction cascade in a reciprocal fashion to each of the instances described above. For example, induction of labor might be achieved by administering a compound which increases the levels of phosphorylated ERK in a pregnant mammal, relative to an otherwise matched pregnant mammal-to which the compound has not been administered. Said compound may activate kinase activity, and the kinase activity activated may be that of a MAPKK. In a preferred embodiment, the MAPKK activated is selected from the group consisting of MEK-1 and MEK-2.

The instant invention also relates to a method for inducing uterine contractions by administering an effective amount of a compound which increases the levels of phosphorylated CaD. Phosphorylation may occur at a C-terminal serine residue which is known in the art to become phosphorylated at the onset of labor. Administration of said compound may activate a kinase, and in a preferred embodiment, the kinase activated is ERK. Phosphorylation may also occur at an ERK-independent phosphorylation site by a kinase such as PAK and/or CaM kinase II. Said compound may activate MAPKK activity, and in a preferred embodiment, the MAPKK activated is selected from the group consisting of MEK-1 and MEK-2. The downstream effect of phosphorylating MAPKK activity may instead be mimicked by a phosphorylation-independent mechanism whereby administration of a compound would activate the binding of CaM to CaD. Such a method would also be effective in inducing uterine contractions in a pregnant mammal.

Conversely, a compound which inhibits an endogenous phosphatase may be administered for increasing the levels of phosphorylated CaD and/or ERK. Inhibition of a phosphatase would have the same effect as activation of a kinase, provided that both enzymes act on the same target molecule. Increasing the levels of phosphorylated CaD and/or ERK by inhibiting a phosphatase may be effective in inducing uterine contractions in a pregnant mammal. The phosphatase targeted may be an ERK phosphatase, a CaD phosphatase, or an upstream phosphatase in the MAPK phosphorelay cascade. In a preferred embodiment, administration of the compound targeting the phosphatase results in increased levels of caldesmon phosphorylated at a C-terminal serine residue or ERK phosphorylation site.

The present invention also relates to a method for inducing uterine contractions in a pregnant mammal by increasing the levels of uterine phosphorylated LC20. An increase in the levels of uterine phosphorylated LC20 is associated with the activation of ERK and the onset of labor. Administration of a compound which activates kinase activity, thereby resulting in an increase in the levels of uterine phosphorylated LC20, may be utilized in the induction of uterine contractions. In a preferred embodiment, the kinase activity which is activated is the kinase activity of MAPKK. Activators of other kinases may be administered with regard to the present invention for increasing levels of uterine phosphorylated LC20.

Administration of a compound which would increase uterine levels of ERK would also likely induce uterine contractions. An increase in uterine ERK protein expression, for example, would cause an increase in the amount of ERK available for phosphorylation by a kinase. The result would be an in an inherent increase in the levels of phosphorylated, and hence activated, ERK.

Compounds for use in the present invention would be administered in a physiologically acceptable carrier in a therapeutically effective dose. Said compound or compounds may be administered alone or in combination with other therapies to prolong or abbreviate a natural course of pregnancy.

Previously identified inhibitors of the ERK MAPK pathway may be utilized in the methods disclosed herein. Examples of known MEK inhibitors include U0126 and PD98059. Examples of ERK inhibitors include pyrazole compositions and isoxazole compositions, the disclosure of isoxazole compositions herein incorporated by reference (Hale et al., (2002) U.S. Pat. No 6,495,582). Identification of additional inhibitors may be accomplished by one of skill in the art through routine experimentation involving in vitro phosphorylation assays. For example, a compound library may be screened with a cascade assay that measures the phosphorylation of a substrate in the presence of purified kinase proteins. Order-of-addition experiments may be utilized to determine which kinase in the MAPK phosphorelay system is inhibited. A potential inhibitor may be added to a tube initially containing an upstream kinase in the MAPK/ERK cascade, buffer salts, and unlabelled ATP. Said upstream kinase is the kinase whose activity is to be assayed. The upstream kinase may be a MAPKK, MAPK, or ERK. If the upstream kinase to be assayed normally activates a downstream kinase, purified downstream kinase is then added along with a proper substrate and [γ-³²P]ATP. If said upstream kinase is MAPKK or MAPK, then said downstream kinase is MAPK or ERK, respectively. If said upstream kinase is ERK, then only a proper substrate and [γ-³²P]ATP need be added. Proper substrates for MEK and ERK may be ERK and CaD, respectively. Transfer of the [γ-³²p] to the substrate may then be determined. In a second order-of-addition experiment, the potential inhibitor compound may be added to a tube containing both upstream and downstream purified kinase proteins, buffer salts, and unlabeled ATP. A proper substrate for the downstream kinase may then be added and transfer of the [γ-³²p] to the substrate determined. Transfer in the second but not the first order-of-addition experiment is indicative that the compound is an inhibitor of said upstream kinase. This method was utilized in the identification of the MEK inhibitor PD 098059. Variations on this assay may be made, such as omission of the addition of labeled [γ-³²P]ATP if another detection method such as antibody recognition is available. Order-of-addition experiments need not be done, as the inability of a kinase to transfer of a labeled phosphate to its substrate may serve as a direct indicator of kinase inhibition.

Alternatively, a cell-based screen may be utilized for identification of compounds as inhibitors of the ERK MAPK pathway. Tissue culture cells may be treated with potential inhibitors prior to stimulation with PMA and PHA. Cells may thereafter be pelleted, washed with appropriate buffer, and lysed by the addition of detergent and/or passage through a fine-gauged needle. Cellular debris may then be removed and the supernatant immunoprecipitated with an antibody to the desired kinase whose inhibition is to be assayed. Immunoprecipitates may then be assayed for activity via their ability to transfer a labeled phosphate group to a specific substrate in an in vitro kinase assay. The inability of a particular kinase to transfer a labeled phosphate group to its substrate may serve as a direct indicator of kinase inhibition. This method was utilized in the identification of the MEK inhibitor U0126.

### EXEMPLIFICATION

### Example 1

### ERK Inhibition Delays the Onset of Labor and Demonstrates a Cause and Effect Relationship Between ERK Phosphorylation and Contractility

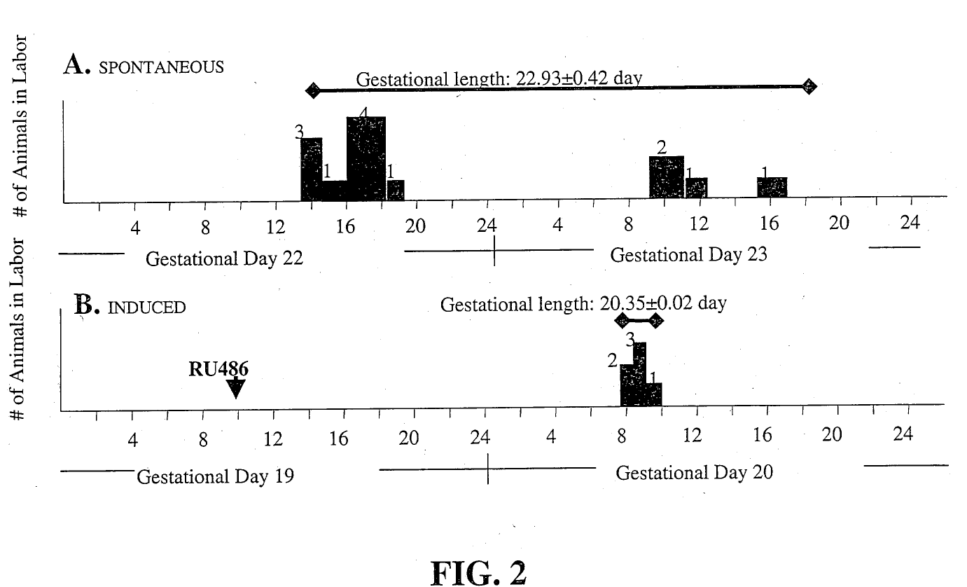

RU486 induces a tightly controlled model of preterm labor in Sprague-Dawley rats. Under standard housing conditions, control Sprague-Dawley pregnant rats (n=13) went into spontaneous labor at a gestational stage of 22.93±0.42 days (Mean±SD). The individual deliveries showed a sizable range in their occurrence with a cluster in the afternoon of the 22^{nd} day of pregnancy and another cluster in the morning of the 23^{rd}-day of pregnancy (FIG. 2A). The average length of pregnancy in these rats was consistent with a previous report in the same model (Telleria et al., J. Steroid Biochem. Mol. Biol. 52, 567-573 (1995)).

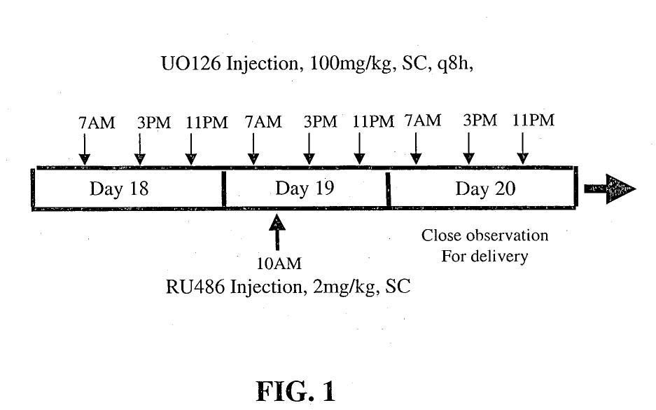

The antiprogesterone RU486 has been used to induce a reliable model for preterm labor/birth in pregnant rats (Cabrol et al., Prostaglandins 42, 71-79 (1991); Garfield et al., Am. J. Obstet. Gynecol. 157,1281-1285 (1987); Telleria et al., J. Steroid Biochem. Mol. Biol. 52, 567-573 (1995)). In the present study, pregnant rats were injected subcutaneously with RU486 at 10 am of the nineteenth day of gestation (FIG. 1). All rats (n=6) gave preterm birth 22.25±0.24 hrs after injection (FIG. 2B), thus delivery occurred in a much more narrow range, 20.35±0.02 days (Mean±SD), with a far smaller SD than that for spontaneous labor.

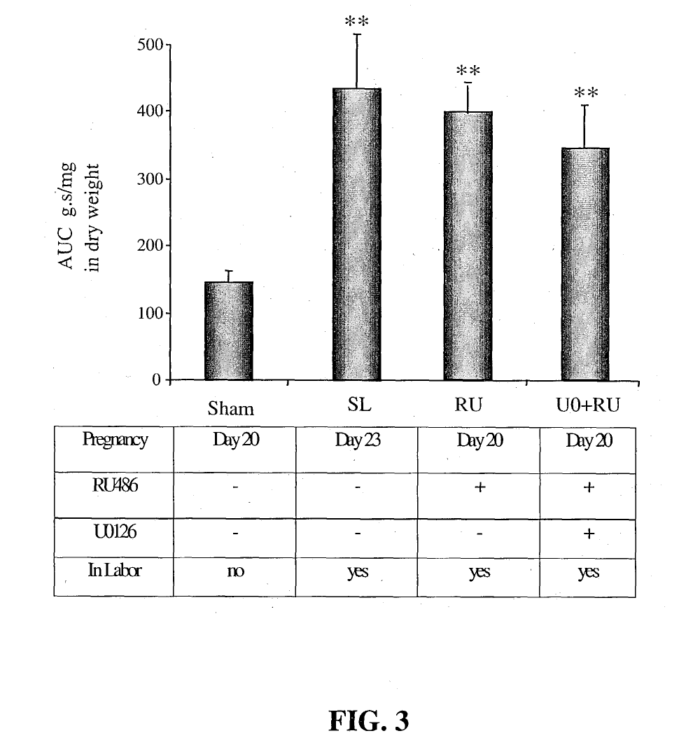

Strips from RU486 treated animals display increased myometrial contractility in vitro. The fact that RU486 can induce preterm labor implies an effect on myometrial contractility; however, whether this increased contractility is associated with chronic changes in basal contractility that persist in vitro in the absence of the hormonal milieu of the intact rats has not been previously shown. Thus, the in vitro area under force curves (AUC) in the sham group, RU group and spontaneous labor (SL) group were compared. As described previously (Li et al., Am. J. Physiol. Regul. Integr. Comp. Physiol. 284, R192-199 (2003)), the in vitro myometrial strips were relatively quiescent on day 20 of pregnancy (FIG. 3, sham group) displaying a lower AUC after normalization to tissue dry weight (153.21±15.22 g.s./mg,) compared to myometrial strips from rats in spontaneous labor (FIG. 3, SL group). In vitro uterine contractility increased dramatically after RU486 treatment (FIG. 3, RU group, AUC 381.49±44.92 g.s./mg), comparable to that during spontaneous labor (AUC 420.14±92.36 g.s./mg, p=0.69).

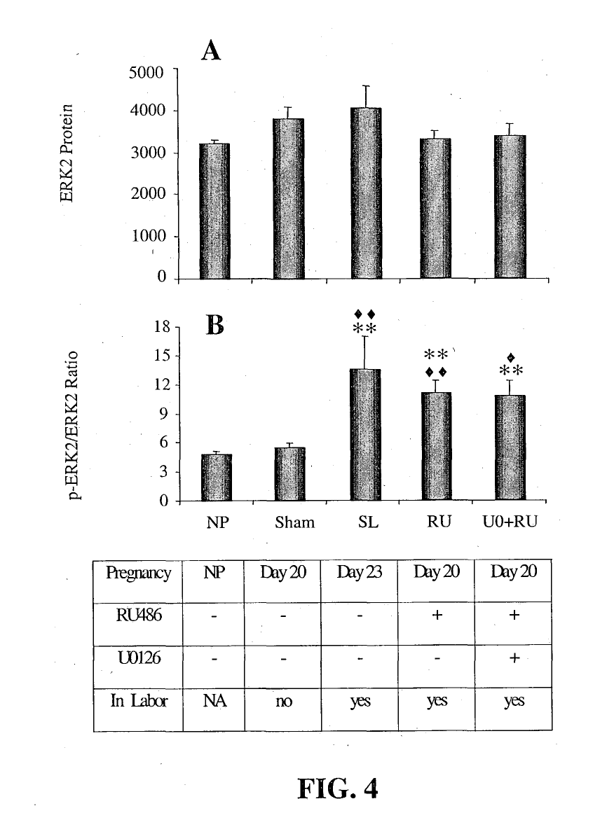

ERK2 is activated during RU486-inducedpreterm labor. Spontaneous labor is associated with phosphorylation, and hence, the activation of ERK2 and subsequent phosphorylation of CaD, and these changes persist in vitro (Li et al., Am. J. Physiol. Regul. lntegr. Comp. Physiol. 284, R192-199 (2003)). The question arises as to whether RU486-induced preterm labor causes the same changes in basal ERK2 and CaD phosphorylation as occurs in spontaneous labor. As is shown in FIG. 4A, ERK2 protein levels did not change throughout pregnancy into spontaneous labor; however, ERK2 phosphorylation significantly increased during RU486-induced labor (FIG. 4B, RU group) to a level comparable to that seen in spontaneous labor (FIG. 4B, SL group).

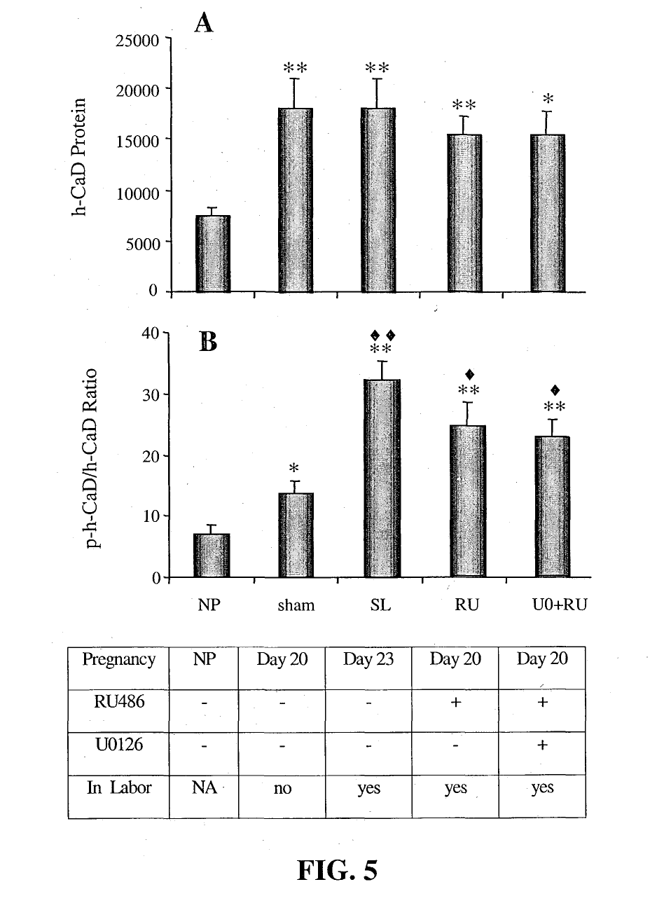

h-CaD phosphorylation is increased during RU486-induced preterm labor. The effects of RU486 on alterations at the level of the contractile apparatus have not been reported. To explore the subcellular mechanism(s) of the effect of RU486 on myometrial contractility, the protein and phosphorylation levels of CaD were examined. Compared to nonpregnant myometrium (NP), CaD protein content is significantly increased during pregnancy at gestational day 20 in protein matched samples (FIG. 5A, D20) as described previously (Li et al., Am. J. Physiol. Regul. Integr. Comp. Physiol. 284, R192-199 (2003)). The phospho-CaD antibody used is specific for CaD phosphorylation at an ERK site, Ser⁷⁸⁹ (D'Angelo et al., J. Biol. Chem. 274, 30115-30121 (1999)). After normalization for the increase in CaD protein levels, p-CaD signals were significantly increased in strips from rats in which RU486 was used to induce preterm labor (FIG. 5B, RU group). There was no difference in the levels of CaD phosphorylation, as measured by this method, between protein matched samples from RU486-induced labor versus spontaneous labor (FIG. 5B, SL group).

Basal 20-kDa myosin light chain phosphorylation (LC20-P) levels increased during RU486-inducedpreterm labor. Smooth muscle contraction can be regulated both by pathways that terminate on the thin filament and those that terminate on the thick filament. To the inventor's knowledge, it is not possible to measure consistent levels of LC20-P in quick frozen rat myometrial muscles because the exposure to cold buffers contracts the muscles and triggers a contraction response, presumably due to released Ca from intracellular store. For this reason, we have previously permeabilized the muscles with alpha-toxin and clamped Ca at resting level (pCa7). A small, but significant, increase in basal 20 kDa myosin light chain (LC20-P) at pCa7 occurs during spontaneous labor and persists in vitro. Thus, the question arises as to whether a similar biochemical profile is seen in vitro for LC20-P in strips from RU486-treated animal. LC20-P levels were significantly increased from 11.81±1.73 mol phosphate/mol LC20 (in sham group) to 17.2±1.36 in myometrium during RU486-induced preterm labor (p<0.05, n=4 or 5 in each group). A LC20-P level of 39.68±3.33 in 51 mM KCI stimulated (for 4 min) myometrial strips was used as a positive control. In general, an increase in basal LC20 phosphorylation would tend to increase contractility during labor.

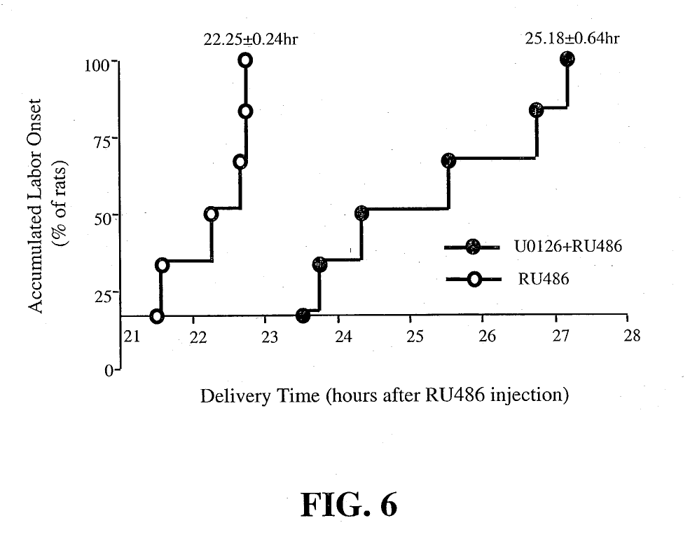

The MEK inhibitor U0126 delays RU486-induced preterm labor in the rats. To determine if there is a cause and effect relationship between activation of the ERK/CaD pathway and the onset of labor contraction, pregnant rats were pretreated with the MEK inhibitor U0126 prior to the administration of RU486 (see methods for details). The compound U0126 {1,4-diamino-2,3-dicyano-1,4-bis (2-aminophenylthio) butadiene} is a potent and specific inhibitor of the mitogen-activated protein kinase kinase (MAPKK) members, MEK-1 and MEK-2. Both are the upstream kinases for ERK1/ERK2 (Cobb et al., Prog. Biophys. Mol. Biol. 71, 479-500 (1999); Favata et al., J. Biol. Chem. 273, 18623-18632 (1998)). ERK inhibitors PD98059 and U0126 have been studied extensively in vitro. However, relative few studies have used ERK inhibitors in vivo because of the low potency of PD98059 and expense of U0126 for chronic treatment (Namura et al., Proc. Natl. Acad. Sci. USA 98, 11569-11574 (2001); Strohm et al., J. Cardiovasc. Pharmacol. 36, 218-229 (2000)). Treatment with U0126 delayed the onset of parturition in a statistically significant manner to an average of 25.18±0.64 hrs after RU486 administration (FIG. 6, p<0.01). As is seen in FIG. 6, there is no overlap of the labor onset times between the RU group and the U0+RU group. Interestingly, despite the fact that U0126 delayed RU486-induced preterm labor, once labor commenced, the myometrial AUC (FIG. 3, U0+RU group), p-ERK (FIG. 4, U0+RU group) and p-CaD levels (FIG. 5, U0+RU group) were indistinguishable between those from laboring animals treated with RU486 or those in spontaneous labor.

Effect of U0126 on Fetal Weight. It is worth noting that even though ERKs are present ubiquitously in most cells of the body; no obvious signs of toxicity to the mothers or pups were observed in this rat model. There was no difference in daily weight gain or general behavior in pregnant rats between the U0+RU group and the RU group (data not shown). U0126 treatment did not affect fetal weight (Table 1).

TABLE 1
Comparison of Fetal Weights (grams)

| Groups | Sham | RU | U0 + RU |
|---|---|---|---|
| Fetal weight | 2.37 ± 0.047 (38) | 2.46 ± 0.068 (16) | 2.46 ± 0.036 (31) |
| (n) | | | |
| Fetal number/ | 12.2 | 13.4 | 12.3 |
| rat | | | |
| Gestation (day) | 20 | 20 | 20 |

### METHODS OF THE INVENTION

Animals and treatment groups. All procedures were approved by our institutional animal care and use committee (IACUC). Sprague-Dawley nonpregnant and time-mated, primigravid pregnant rats (day 1=sperm positive, Taconic, Germantown, N.Y.) were used for the experiments. They were housed in a constant-temperature room with a 12-hr light and 12-hr dark cycle. Food and water were available ad libitum. The rats were divided into five groups: nonpregnant (NP), sham, RU486 treated (RU), U0126+RU486 treated (U0+RU) and spontaneous labor (SL). The sequential scheme of the experiment is illustrated in FIG. 1.

Induction of preterm labor: In the RU group, rats (n=6) were treated with RU486 subcutaneously (SC) at dose of 2 mg/kg (concentration of 3 mg/ml in sunflower seed oil) at 10 am on day 19 of pregnancy. Then the rats were closely observed for signs of labor on day 20 of pregnancy.

U0126 treatment: In the U0+RU group, 18-day pregnant rats (n=6) were treated with U0126 at dose of 100 mg/kg, SC, q8h (7 am, 3 pm and 11 pm). U0126 was dissolved at a concentration of 200 mg/ml in DMSO. On day 19 of pregnancy, the rats were administrated RU486 at 10 am for preterm labor induction. The U0126 treatment continued till the onset of labor.

Sham control: 18-day pregnant rats (n=6) were injected subcutaneously with the same amount of DMSO or sunflower seed oil in the regime described for the U0+RU group. The rats were euthanized via CO₂ inhalation at 11 am of day 20 pregnancy for sample collection to match the average stage of gestation at which the U0+RU treated rats delivered.

Tissue preparation and force recording. For the collection of in-labor uterine smooth muscle samples, rats were closely observed at 15-30 minute intervals. The delivery of the first pup was defined as the onset of labor. Rats were euthanized by carbon dioxide inhalation followed by cervical dislocation. All products of conception were removed from the uterine wall and placed on ice. The blotted weight of each pup was recorded. Excised uteri were immersed immediately into oxygenated Krebs solution at room temperature. The composition of Krebs solution was (mM): 120 NaCl, 5.9 KCI, 11.5 Dextrose, 25 NaHCO₃, 1.2 NaH₂PO₄.H₂O, 1.2 MgCl₂.6H₂O, and 2.5 CaCl₂ Approximate 8×2 mm (LxW) whole thickness uterine strips oriented parallel to the long axis of the longitudinal muscle bundles were dissected under a dissection microscope (Olympus VM, Japan).

Isometric force was recorded at 37° C. as previously described (Li et al., Am. J. Physiol. Regul. Integr. Comp. Physiol. 284, R192-199 (2003); Morgan and Gangopadhyay, J. Appl. Physiol. 91, 953-962 (2001); Szal et al., Am. J. Physiol. 267, E77-87 (1994)). All myometrial strips were gradually stretched to the optimal length (Lo) with respect to spontaneous contractions (Herlihy and Murphy, Circ. Res. 33, 275-283 (1973)). The contractile activity was digitalized with MacLab/8e®, Chart v3.5.4 (AD Instrument Ltd., Castle Hill, Australia) and normalized for tissue dry weight. The area under the curve (AUC) parameter was obtained by integrating the force signal over a 15 minute time period.

Western blot analysis. The muscle strips were quick-frozen in a dry ice acetone/DTT/TCA slurry at the end of force recording experiment. Samples were homogenized in a buffer containing 20 mM MOPS, 4% SDS, 1% Triton, 10% glycerol, 10 mM DTT, 20 mM β-glycerophosphate, 5.5 μM leupeptin, 5.5 μM pepstatin A, 20 KIU aprotinin, 2 mM Na₃VO₄, 1 mM NaF, 20 μM 4-(2-aminorthyl) benzenesulphonyl fluoride (AEBSF) and 100 μM ZnCl₂. Protein-matched samples were SDS-PAGE electrophoresed, transferred to Immobilon-P membrane (Millipore, Bedford, Mass.), and subjected to immunostaining using the appropriate antibody. Blots were visualized with a SuperSignal West Pico Peroxide Solution (Pierce, Rockford, Ill.). The images were detected with a chemiluminescence screen and quantified with a BioRad Molecular Imager and Multi-Analyst software.

Measurements of myosin light chain 20 phosphorylation (LC20-P). Tissue preparation was as described above. After equilibration and stretching, the myometrial strips were permeabilized with 20 μg/ml alpha-toxin (List Biological Lab, Campbell, Calif.) at 34° C. for 30 min. The intracellular Ca2+ concentration was clamped at pCa7.0 for 12 min to prevent an artifactual triggering of contractions in response to the freezing solution. The measurement of LC20-P was performed by glycerol urea gels and western blots as previously published (Je et al., J. Physiol. 537, 567-577 (2001)). Moles phosphate per mole myosin light chain was calculated by dividing the density of the phosphorylated band by the sum of densities of phosphorylated plus the unphosphorylated bands.

Chemicals and antibodies. Mifepristone (RU486; 11 β-[4-Dimethylamino] phenyl-17β-hydroxy-17-[1-propynyl] estra-4,9-dien-3-one) is a progesterone receptor antagonist and a product of Sigma. U0126 (1,4-diamino-2,3-dicyano-1,4-bis [2-aminophenylthio] butadiene) is a selective inhibitor of MAP kinase kinase (MEK1/2). It was a generous gift from DuPont Pharmaceuticals Company (Wilmington, Del.). The phospho-p44/42 MAP kinase antibody (1:2000) and p44/42 MAP kinase antibody (1:1,500) were purchased from Cell Signaling Technology (Beverly, Mass.). The CaD polyclonal antibody (1:30,000) was raised against full-length human myometrial CaD and was a gift from Dr. K. Mabuchi (Boston Biomedical Research Institute, Watertown, Mass.). The anti-phospho CaD antibody (1:500, a gift from Dr. L. Adam, Bristol Myers Squibb, N.J. or Upstate, Lake Placid, N.Y.) was produced against a phosphopeptide containing the CaD sequence surrounding the Ser⁷⁸⁹ ERK phosphorylation site (D'Angelo et al., J. Biol. Chem. 274, 30115-30121 (1999)). The monoclonal anti-myosin light chain antibody (1:2,500) was a product of Sigma. General laboratory reagents were of analytical grade or better and were purchased from Sigma or Fisher Scientific.

Statistics. Data are presented as mean±SEM unless otherwise stated. The n values represent the number of animals used in the experiments. Data were analyzed by ANOVA followed by Bonferroni's post-test or by Student t test. p value<0.05 was considered statistically significant.

1. A method for preventing or delaying preterm uterine contractions in a pregnant mammal, the method comprising administering an effective amount of a compound which inhibits kinase activity, thereby resulting in decreased levels of phosphorylated ERK in the pregnant mammal, relative to an otherwise matched pregnant mammal to which the compound has not been administered.

2. The method of claim 1 wherein the compound is an inhibitor of a mitogen-activated protein kinase kinase (MAPKK).

3. The method of claim 2 wherein the mitogen-activated protein kinase kinase is selected from the group consisting of MEK-1 and MEK-2.

4. The method of claim 2 wherein the inhibitor of a mitogen-activated protein kinase kinase (MAPKK) is U0126.

5. A method for preventing or delaying preterm uterine contractions in a pregnant mammal, the method comprising administering an effective amount of a compound which inhibits kinase activity, thereby resulting in decreased levels of phosphorylated caldesmon in the pregnant mammal, relative to an otherwise matched pregnant mammal to which the compound has not been administered.

6. The method of claim 5 wherein the administration of the compound which inhibits kinase activity results in decreased levels of caldesmon phosphorylated at a C-terminal serine residue.

7. The method of claim 6 wherein the kinase activity inhibited is the kinase activity of ERK.

8. The method of claim 5 wherein the kinase activity inhibited is the kinase activity of a kinase selected from the group consisting of PAK and CaM kinase II.

9. The method of claim 5 wherein the compound is an inhibitor of a mitogen-activated protein kinase kinase (MAPKK).

10. The method of claim 9 wherein the mitogen-activated protein kinase kinase is selected from the group consisting of MEK-1 and MEK-2.

11. The method of claim 9 wherein the inhibitor of a mitogen-activated protein kinase kinase (MAPKK) is U0126.

12. A method for preventing or delaying preterm uterine contractions in a pregnant mammal, the method comprising administering an effective amount of a compound which inhibits the binding of calmodulin to caldesmon.

13. A method for preventing or delaying preterm uterine contractions in a pregnant mammal, the method comprising administering an effective amount of a compound which activates a phosphatase enzyme, thereby resulting in decreased levels of phosphorylated caldesmon in the pregnant mammal, relative to an otherwise matched pregnant mammal to which the compound has not been administered.

14. The method of claim 13 wherein the administration of the compound results in decreased levels of caldesmon phosphorylated at a C-terminal serine residue.

15. A method for preventing or delaying preterm uterine contractions in a pregnant mammal, the method comprising administering an effective amount of a compound which activates a phosphatase enzyme, thereby resulting in decreased levels of phosphorylated ERK in the pregnant mammal, relative to an otherwise matched pregnant mammal to which the compound has not been administered.

16. A method for preventing or delaying preterm uterine contractions in a pregnant mammal, the method comprising administering an effective amount of a compound which inhibits kinase activity, thereby resulting in decreased levels of uterine phosphorylated myosin light chain (LC20) in the pregnant mammal, relative to an otherwise matched pregnant mammal to which the compound has not been administered, the kinase activity of the RhoA/Rho-kinase being specifically excluded.

17. The method of claim 16 wherein the compound is an inhibitor of a mitogen-activated protein kinase kinase (MAPKK).

18. The method of claim 17 wherein the mitogen-activated protein kinase kinase is selected from the group consisting of MEK-1 and MEK-2.

19. The method of claim 17 wherein the inhibitor of a mitogen-activated protein kinase kinase (MAPKK) is U0126.

20. A method for inducing uterine contractions in a pregnant mammal, the method comprising administering an effective amount of a compound which activates kinase activity, thereby resulting in increased levels of phosphorylated ERK in the pregnant mammal, relative to an otherwise matched pregnant mammal to which the compound has not been administered.

21. The method of claim 20 wherein the compound is an activator of a mitogen-activated protein kinase kinase (MAPKK).

22. The method of claim 21 wherein the mitogen-activated protein kinase kinase is selected from the group consisting of MEK-1 and MEK-2.

23. A method for inducing uterine contractions in a pregnant mammal, the method comprising administering an effective amount of a compound which activates kinase activity, thereby resulting in increased levels of phosphorylated caldesmon in the pregnant mammal, relative to an otherwise matched pregnant mammal to which the compound has not been administered.

24. The method of claim 23 wherein the administration of the compound which activates kinase activity results in increased levels of caldesmon phosphorylated at a C-terminal serine residue.

25. The method of claim 24 wherein the kinase activity activated is the kinase activity of ERK.

26. The method of claim 23 wherein the kinase activity activated is the kinase activity of a kinase selected from the group consisting of PAK and CaM kinase II.

27. The method of claim 23 wherein the compound is an activator of the mitogen-activated protein kinase kinase (MAPKK).

28. The method of claim 27 wherein the mitogen-activated protein kinase kinase is selected from the group consisting of MEK-1 and MEK-2.

29. A method for inducing uterine contractions in a pregnant mammal, the method comprising administering an effective amount of a compound which activates the binding of calmodulin to caldesmon.

30. A method for inducing uterine contractions in a pregnant mammal, the method comprising administering an effective amount of a compound which inhibits a phosphatase enzyme, thereby resulting in increased levels of phosphorylated caldesmon in the pregnant mammal, relative to an otherwise matched pregnant mammal to which the compound has not been administered.

31. The method of claim 30 wherein the administration of the compound results in increased levels of caldesmon phosphorylated at a C-terminal serine residue.

32. A method for inducing uterine contractions in a pregnant mammal, the method comprising administering an effective amount of a compound which inhibits a phosphatase enzyme, thereby resulting in increased levels of phosphorylated ERK in the pregnant mammal, relative to an otherwise matched pregnant mammal to which the compound has not been administered.

33. A method for inducing uterine contractions in a pregnant mammal, the method comprising administering an effective amount of a compound which activates kinase activity, thereby resulting in increased uterine levels of phosphorylated myosin light chain (LC20) in the pregnant mammal, relative to an otherwise matched pregnant mammal to which the compound has not been administered, the kinase activity of the RhoA/Rho-kinase being specifically excluded.

34. The method of claim 33 wherein the compound is an activator of a mitogen-activated protein kinase kinase (MAPKK).

35. The method of claim 34 wherein the mitogen-activated protein kinase kinase is selected from the group consisting of MEK-1 and MEK-2.
